(19) United States
(12) Patent Application Publication
(10) Pub. No.: US 2017/0369396 A1
(43) Pub. Date: Dec. 28, 2017

(54) ISOPARAFFIN-OLEFIN ALKYLATION

(71) Applicant: ExxonMobil Research and Engineering Company, Annandale, NJ (US)

(72) Inventors: Jihad M. DAKKA, Whitehouse Station, NJ (US); Matthew S. METTLER, Somerville, NJ (US); Jeffrey M. FITT, The Woodlands, TX (US); Ivy D. JOHNSON, Lawrenceville, NJ (US); Charles M. SMITH, Princeton, NJ (US)

(21) Appl. No.: 15/610,745

(22) Filed: Jun. 1, 2017

Related U.S. Application Data
(60) Provisional application No. 62/353,687, filed on Jun. 23, 2016.

(51) Int. Cl. C07C 2/58; B01J 29/70; C10L 1/16; C10L 10/10; B01J 37/00; B01J 37/08

(52) U.S. Cl. CPC C07C 2/58; B01J 37/0018; B01J 37/08; C10L 1/1608; C10L 10/10; B01J 29/7038; C07C 2521/04; C07C 2529/70; C10L 2270/023; C07C 2521/08

(57) ABSTRACT

In a process for isoparaffin-olefin alkylation, a feed comprising at least one olefin and at least one isoparaffin is contacted under alkylation conditions in the presence of a solid acid catalyst comprising a crystalline microporous material of the MWW framework type to produce an alkylated product. The alkylated product comprises a C₈₋ fraction, which is useful as a gasoline blending stock, and a C₉₊ fraction, which is separated from the alkylated product and at least partially recycled to the alkylation step.

### CROSS-REFERENCE TO RELATED APPLICATIONS

This application claims the benefit of U.S. Provisional Application No. 62/353,687, filed on Jun. 23, 2016, the entire contents of which is incorporated herein by reference.

### FIELD

The present disclosure relates to a process for isoparaffin-olefin alkylation.

### BACKGROUND

Alkylation is a reaction in which an alkyl group is added to an organic molecule. Thus an isoparaffin can be reacted with an olefin to provide an isoparaffin of higher molecular weight. Industrially, the concept depends on the reaction of a C₂ to C₅ olefin, normally 2-butene, with isobutane in the presence of an acidic catalyst to produce a so-called alkylate. This alkylate is a valuable blending component in the manufacture of gasoline due not only to its high octane rating but also to its sensitivity to octane-enhancing additives.

Industrial isoparaffin-olefin alkylation processes have historically used hydrofluoric or sulfuric acid catalysts under relatively low temperature conditions. The sulfuric acid alkylation reaction is particularly sensitive to temperature, with low temperatures being favored to minimize the side reaction of olefin polymerization. Acid strength in these liquid acid catalyzed alkylation processes is preferably maintained at 88 to 94 weight percent by the continuous addition of fresh acid and the continuous withdrawal of spent acid. The hydrofluoric acid process is less temperature sensitive and the acid is more easily recovered and purified.

A general discussion of sulfuric acid alkylation can be found in a series of three articles by L. F. Albright et al., “Alkylation of Isobutane with C₄ Olefins”, 27 Ind. Eng. Chem. Res., 381-397, (1988). For a survey of hydrofluoric acid catalyzed alkylation, see 1 Handbook of Petroleum Refining Processes 23-28 (R. A. Meyers, ed., 1986). An overview of the entire technology can be found in “Chemistry, Catalysts and Processes of Isoparaffin-Olefin Alkylation —Actual Situation and Future Trends, Corma et al., Catal. Rev.—Sci. Eng. 35(4), 483-570 (1993).

Both sulfuric acid and hydrofluoric acid alkylation share inherent drawbacks including environmental and safety concerns, acid consumption, and sludge disposal. In addition, alkylation processes catalyzed by hydrofluoric and sulfuric acids are generally feed restricted in that only certain short chain (C₅ and below) olefins and C₄ isoparaffins can be used. Otherwise, the activity and stability of the catalyst are adversely affected.

Research efforts have, therefore, been directed towards developing alkylation catalysts which are equally as effective as sulfuric or hydrofluoric acids but which avoid many of the problems associated with these two acids. In particular, research has been focused on the development of solid, instead of liquid, acid alkylation catalyst systems.

For example, U.S. Pat. No. 3,644,565 discloses alkylation of a paraffin with an olefin in the presence of a catalyst comprising a Group VIII noble metal present on a crystalline aluminosilicate zeolite having pores of substantially uniform diameter from about 4 to 18 angstrom units and a silica to alumina ratio of 2.5 to 10, such as zeolite Y. The catalyst is pretreated with hydrogen to promote selectivity.

However, the development of a satisfactory solid acid replacement for hydrofluoric and sulfuric acid has proved challenging. For example, U.S. Pat. No. 4,384,161 describes a process of alkylating isoparaffins with olefins to provide alkylate using a large-pore zeolite catalyst capable of absorbing 2,2,4-trimethylpentane, for example, ZSM-4, ZSM-20, ZSM-3, ZSM-18, zeolite Beta, faujasite, mordenite, zeolite Y and the rare earth metal-containing forms thereof, and a Lewis acid such as boron trifluoride, antimony pentafluoride or aluminum trichloride. The addition of a Lewis acid is reported to increase the activity and selectivity of the zeolite, thereby effecting alkylation with high olefin space velocity and low isoparaffin/olefin ratio. According to the ‘161 patent, problems arise in the use of solid catalysts alone in that they appear to age rapidly and cannot perform effectively at high olefin space velocity.

As new solid acid catalysts have become available, they have been routinely screened for their efficacy in isoparaffin-olefin alkylation. For example, U.S. Pat. No. 5,304,698 describes a process for the catalytic alkylation of an olefin with an isoparaffin comprising contacting an olefin-containing feed with an isoparaffin-containing feed with a crystalline microporous material selected from the group consisting of MCM-22, MCM-36, and MCM-49 under alkylation conversion conditions of temperature at least equal to the critical temperature of the principal isoparaffin component of the feed and pressure at least equal to the critical pressure of the principal isoparaffin component of the feed.

Despite extensive research, there remains an unmet need for an improved isoparaffin-olefin alkylation process that is catalyzed by a solid acid catalyst but approaches or exceeds the activity, stability and alkylate yield of existing liquid phase processes.

### SUMMARY

According to the present disclosure, it has now been found that MWW framework-type zeolites exhibit unexpectedly high activity and selectivity as catalysts for isoparaffin-olefin alkylation including with feeds containing significant amounts of heavy (C₁₀₊) components such as those generated as by-products of the alkylation process. Although the reasons for this result are not fully understood, it is believed that the heavy components are cracked in the presence of the MWW zeolite catalyst to produce light olefins and paraffins which can react to generate additional alkylate product. Not only does this allow increased alkylate yield but removing and recycling the heavy by-products reduces catalyst aging and allows the process to be operated at lower pressure thereby reducing capital and operating costs.

In one aspect, the present disclosure resides in a process for the catalytic alkylation of an olefin with an isoparaffin, the process comprising:

(a) contacting a feed comprising at least one olefin and at least one isoparaffin with a solid acid catalyst under alkylation conditions effective for reaction between the olefin and the isoparaffin to produce an alkylated product, wherein the solid acid catalyst comprises a crystalline microporous material of the MWW framework type,

(b) separating a C₁₀₊ fraction from the alkylated product; and

(c) recycling at least part of the C₁₀₊ fraction to the contacting (a).

### BRIEF DESCRIPTION OF THE DRAWINGS

FIG. 1 is a graph of isooctene conversion against time on stream (days) for the MCM-49 catalyst of Example 1 in the alkylation of a premixed isobutane/isooctene feed at various temperatures according to the process of Example 2.

FIG. 2 is a graph of % production of 2,2,4-trimethylpentane against total trimethylpentane production for the MCM-49 catalyst of Example 1 in the alkylation of a premixed isobutane/isooctene feed at various temperatures according to the process of Example 2.

FIG. 3 is a graph of product selectivity against time on stream (days) for (a) the C₈ product fraction, (b) the C₉₊ product fraction and (c) the cracking product (C₅, C₆ and C₇) obtained in the process of Example 2.

FIG. 4 is a graph of butene conversion against the material balance (MB) number based on online GC analysis taken every 3 hrs for (a) a sand blank and (b) an REX catalyst in the alkylation of a premixed isobutane/butene feed according to the process of Example 3.

### DETAILED DESCRIPTION OF THE EMBODIMENTS

Disclosed herein is a process for isoparaffin-olefin alkylation, in which an olefin-containing feed is contacted with an isoparaffin-containing feed under alkylation conditions in the presence of a solid acid catalyst comprising a crystalline microporous material of the MWW framework type to produce an alkylated product. The alkylated product comprises a C₉₋ fraction, which is useful as a gasoline blending stock, and a C₁₀₊ fraction, which is separated from the alkylated product and at least partially recycled to the alkylation step. Surprisingly, it has been found that, using an MWW framework type alkylation catalyst, the recycled C₁₀₊ hydrocarbons are cracked in the alkylation reactor to generate light olefins and isoparaffins, both of which are alkylated to generate additional alkylate product. Moreover, this improvement in alkylate yield is achieved without the rapid deactivation generally experienced in the presence of heavy feeds with homogeneous catalysts, such as sulfuric acid and hydrofluoric acid.

As used herein, the term “Cₙ” compound (olefin or paraffin) wherein n is a positive integer, e.g., 1, 2, 3, 4, 5, etc, means a compound having n number of carbon atom(s) per molecule. The term “Cₙ₊” compound wherein n is a positive integer, e.g., 1, 2, 3, 4, 5, etc, means a compound having at least n number of carbon atom(s) per molecule. The term “Cₙ₋” compound wherein n is a positive integer, e.g., 1, 2, 3, 4, 5, etc, as used herein, means a compound having no more than n number of carbon atom(s) per molecule.

As used herein, the term “crystalline microporous material of the MWW framework type” includes one or more of:
molecular sieves made from a common first degree crystalline building block unit cell, which unit cell has the MWW framework topology. (A unit cell is a spatial arrangement of atoms which if tiled in three-dimensional space describes the crystal structure. Such crystal structures are discussed in the “Atlas of Zeolite Framework Types”, Fifth edition, 2001, the entire content of which is incorporated as reference);
molecular sieves made from a common second degree building block, being a 2-dimensional tiling of such MWW framework topology unit cells, forming a monolayer of one unit cell thickness, preferably one c-unit cell thickness;
molecular sieves made from common second degree building blocks, being layers of one or more than one unit cell thickness, wherein the layer of more than one unit cell thickness is made from stacking, packing, or binding at least two monolayers of MWW framework topology unit cells. The stacking of such second degree building blocks can be in a regular fashion, an irregular fashion, a random fashion, or any combination thereof; and
molecular sieves made by any regular or random 2-dimensional or 3-dimensional combination of unit cells having the MWW framework topology.

Crystalline microporous materials of the MWW framework type include those molecular sieves having an X-ray diffraction pattern including d-spacing maxima at 12.4±0.25, 6.9±0.15, 3.57±0.07 and 3.42±0.07 Angstrom. The X-ray diffraction data used to characterize the material are obtained by standard techniques using the K-alpha doublet of copper as incident radiation and a diffractometer equipped with a scintillation counter and associated computer as the collection system.

Examples of crystalline microporous materials of the MWW framework type include MCM-22 (described in U.S. Pat. No. 4,954,325), PSH-3 (described in U.S. Pat. No. 4,439,409), SSZ-25 (described in U.S. Pat. No. 4,826,667), ERB-1 (described in European Patent No. 0293032), ITQ-1 (described in U.S. Pat. No 6,077,498), ITQ-2 (described in International Patent Publication No. W097/17290), MCM-36 (described in U.S. Pat. No. 5,250,277), MCM-49 (described in U.S. Pat. No. 5,236,575), MCM-56 (described in U.S. Pat. No. 5,362,697), UZM-8 (described in U.S. Pat. No. 6,756,030), UZM-8HS (described in U.S. Pat. No. 7,713,513), UZM-37 (described in U.S. Pat. No. 7,982,084); EMM-10 (described in U.S. Pat. No. 7,842,277), EMM-12 (described in U.S. Pat. No. 8,704,025), EMM-13 (described in U.S. Pat. No. 8,704,023), MIT-1 (described by Luo et al in Chem. Sci., 2015, 6, 6320-6324) and mixtures thereof, with MCM-49 generally being preferred.

In some embodiments, the crystalline microporous material of the MWW framework type employed herein may be an aluminosilicate material having a silica to alumina molar ratio of at least 10, such as at least 10 to less than 50.

In some embodiments, the crystalline microporous material of the MWW framework type employed herein may be contaminated with other crystalline materials, such as ferrierite or quartz. These contaminants may be present in quantities ≦10% by weight, normally ≦5% by weight.

The above molecular sieves may be used in the alkylation catalyst without any binder or matrix, i.e., in so-called self-bound form. Alternatively, the molecular sieve may be composited with another material which is resistant to the temperatures and other conditions employed in the alkylation reaction. Such materials include active and inactive materials and synthetic or naturally occurring zeolites as well as inorganic materials such as clays and/or oxides such as alumina, silica, silica-alumina, zirconia, titania, magnesia or mixtures of these and other oxides. The latter may be either naturally occurring or in the form of gelatinous precipitates or gels including mixtures of silica and metal oxides. Clays may also be included with the oxide type binders to modify the mechanical properties of the catalyst or to assist in its manufacture. Use of a material in conjunction with the molecular sieve, i.e., combined therewith or present during its synthesis, which itself is catalytically active may change the conversion and/or selectivity of the catalyst. Inactive materials suitably serve as diluents to control the amount of conversion so that products may be obtained economically and orderly without employing other means for controlling the rate of reaction. These materials may be incorporated into naturally occurring clays, e.g., bentonite and kaolin, to improve the crush strength of the catalyst under commercial operating conditions and function as binders or matrices for the catalyst. The relative proportions of molecular sieve and inorganic oxide binder may vary widely. For example, the amount of binder employed may be as little as 1 wt %, such as at least 5 wt %, for example at least 10 wt %, whereas in other embodiments the catalyst may include up to 90 wt %, for example up 80 wt %, such as up to 70 wt %, for example up to 60 wt %, such as up to 50 wt % of a binder material.

In one embodiment, the solid acid catalyst employed in the present process is substantially free of any binder containing amorphous alumina. As used herein, the term “substantially free of any binder containing amorphous alumina” means that the solid acid catalyst used herein contains less than 5 wt %, such as less than 1 wt %, and preferably no measurable amount, of amorphous alumina as a binder. Surprisingly, it is found that when the solid acid catalyst is substantially free of any binder containing amorphous alumina, the activity of the catalyst for isoparaffin-olefin alkylation can be significantly increased, for example by at least 50%, such as at least 75%, even at least 100% as compared with the activity of an identical catalyst but with an amorphous alumina binder.

The present alkylation process is suitably conducted at temperatures from about 275° F. to about 700° F. (135° C. to 371° C.), such as from about 300° F. to about 600° F. (149° C. to 316° C.). Operating temperatures typically exceed the critical temperature of the principal component in the feed. The term “principal component” as used herein is defined as the component of highest concentration in the feedstock. For example, isobutane is the principal component in a feedstock consisting of isobutane and 2-butene in an isobutane:2-butene weight ratio of 50:1.

Operating pressure may similarly be controlled to maintain the principal component of the feed in the supercritical state, and is suitably from about 300 to about 1500 psig (2170 kPa-a to 10,445 kPa-a), such as from about 400 to about 1000 psig (2859 kPa-a to 6996 kPa-a). In some embodiments, the operating temperature and pressure remain above the critical value for the principal feed component during the entire process run, including the first contact between fresh catalyst and fresh feed.

Hydrocarbon flow through the alkylation reaction zone containing the catalyst is typically controlled to provide a total liquid hourly space velocity (LHSV) sufficient to convert about 99 percent by weight of the fresh olefin to alkylate product. In some embodiments, olefin LHSV values fall within the range of about 0.01 to about 10 hr⁻¹.

The present isoparaffin-olefin alkylation process can be conducted in any known reactor, including reactors which allow for continuous or semi-continuous catalyst regeneration, such as fluidized and moving bed reactors, as well as swing bed reactor systems where multiple reactors are oscillated between on-stream mode and regeneration mode. Surprisingly, however, it is found that catalysts employing MWW framework type molecular sieves show unusual stability when used in isoparaffin-olefin alkylation even with feeds containing C₅₊ olefins and/or C₅₊ isoparaffins. Thus, MWW-containing alkylation catalysts are particularly suitable for use in simple fixed bed reactors, without swing bed capability. In such cases, cycle lengths (on-stream times between successive catalyst regenerations) in excess of 150 days may be obtained.

Feedstocks useful in the present alkylation process include at least one isoparaffin and at least one olefin. The isoparaffin reactant used in the present alkylation process may have from about 4 to about 8 carbon atoms. Representative examples of such isoparaffins include isobutane, isopentane, 3-methylhexane, 2-methylhexane, 2,3-dimethylbutane, 2,4-dimethylhexane and mixtures thereof, especially isobutane.

The olefin component of the feedstock may include at least one olefin having from 3 to 12 carbon atoms. Representative examples of such olefins include butene-2, isobutylene, butene-1, propylene, ethylene, hexene, octene, and heptene, merely to name a few. In some embodiments, the olefin component of the feedstock is selected from the group consisting of propylene, butenes, pentenes and mixtures thereof. For example, in one embodiment, the olefin component of the feedstock may include a mixture of propylene and at least one butene, especially 2-butene, where the weight ratio of propylene to butene is from 0.01:1 to 1.5:1, such as from 0.1:1 to 1:1. In another embodiment, the olefin component of the feedstock may include a mixture of propylene and at least one pentene, where the weight ratio of propylene to pentene is from 0.01:1 to 1.5:1, such as from 0.1:1 to 1:1.

Isoparaffin to olefin ratios in the reactor feed typically range from about 1.5:1 to about 100:1, such as 10:1 to 75:1, measured on a volume to volume basis, so as to produce a high quality alkylate product at industrially useful yields. Higher isoparaffin:olefin ratios may also be used, but limited availability of produced isoparaffin within many refineries coupled with the relatively high cost of purchased isoparaffin favor isoparaffin:olefin ratios within the ranges listed above.

The olefin-containing feedstock and the isoparaffin-containing feedstock may be mixed prior to being fed to the alkylation reaction zone or may be supplied separately to the reaction zone. In addition, before being sent to the alkylation reaction zone, the isoparaffin and/or olefin may be treated to remove catalyst poisons e.g., using guard beds with specific absorbents for reducing the level of S, N, and/or oxygenates to values which do not affect catalyst stability activity and selectivity.

The product composition of the isoparaffin-olefin alkylation reaction described herein is highly dependent on the reaction conditions and the composition of the olefin and isoparaffin feedstocks. In any event, the product is a complex mixture of hydrocarbons, since alkylation of the feed isoparaffin by the feed olefin is accompanied by a variety of competing reactions including cracking, olefin oligomerization and further alkylation of the alkylate product by the feed olefin. For example, in the case of alkylation of isobutane with C₃-C₅ olefins, particularly 2-butene, the product may comprise about 20 wt % of C₅-C₇ hydrocarbons, 60-65 wt % of octanes, 10-15 wt % of C₉ hydrocarbons and 5-10 wt % C₁₀₊ hydrocarbons. Moreover, using an MWW type molecular sieve as the catalyst, it is found that the process is selective to desirable high octane components so that, in the case of alkylation of isobutane with C₃-05 olefins, the C₆ fraction typically comprises at least 40 wt %, such as at least 70 wt %, of 2,3-dimethylbutane and the C₅ fraction typically comprises at least 50 wt %, such as at least 70 wt %, of 2,3,4 and 2,3,3 and 2,2,4-trimethylpentane.

The product of the isoparaffin-olefin alkylation reaction is fed to a separation system, such as a distillation train, to separate the alkylate product into at least a C₉₋ fraction and a C₁₀₊ fraction. The C₉₋ fraction is recovered for use as a gasoline octane enhancer, while at least part of the C₁₀₊ fraction is recycled to the alkylation step. Surprisingly, it has been found that, using an MWW framework type alkylation catalyst, the recycled C₁₀₊ hydrocarbons are cracked in the alkylation reactor to generate light olefins and isoparaffins, both of which are alkylated to generate additional alkylate product. Moreover, this improvement in alkylate yield is achieved without the rapid deactivation generally experienced in the presence of heavy feeds with homogeneous catalysts, such as sulfuric acid and hydrofluoric acid. or can be recycled to the alkylation reactor to generate more alkylate. In particular, it is found that MWW type molecular sieves are effective to crack the C₁₀₊ fraction to produce light olefins and paraffins which can react to generate additional alkylate product and thereby increase overall alkylate yield.

In some embodiments, the ratio of the weight of C₁₀+ fraction recycled to the alkylation step to the weight of isoparaffin/olefin feed to the alkylation step is from 0.1 to 5, such as from 0.2 to 1.

Depending on the demand for alkylate versus that for distillate, part of the C₁₀₊ fraction can be recovered for use as a distillate blending stock.

The invention will now be more particularly described with reference to the following non-limiting Examples and the accompanying drawings.

### EXAMPLE 1

### Preparation of 80wt % MCM-49/20wt % Alumina Catalyst

80 parts MCM-49 zeolite crystals are combined with 20 parts pseudoboehmite alumina, on a calcined dry weight basis. The MCM-49 and pseudoboehmite alumina dry powder are placed in a muller or a mixer and mixed for about 10 to 30 minutes. Sufficient water and 0.05% polyvinyl alcohol are added to the MCM-49 and alumina during the mixing process to produce an extrudable paste. The extrudable paste is formed into a 1/20 inch quadralobe extrudate using an extruder. After extrusion, the 1/20th inch quadralobe extrudate is dried at a temperature ranging from 250° F. to 325° F. (121 to 163° C.). After drying, the dried extrudate is heated to 1000° F. (538° C.) under flowing nitrogen. The extrudate is then cooled to ambient temperature and humidified with saturated air or steam.

After humidification, the extrudate is ion exchanged with 0.5 to 1 N ammonium nitrate solution. The ammonium nitrate solution ion exchange is repeated. The ammonium nitrate exchanged extrudate is then washed with deionized water to remove residual nitrate prior to calcination in air. After washing the wet extrudate, it is dried. The exchanged and dried extrudate is then calcined in a nitrogen/air mixture to a temperature 1000° F. (538° C.).

### EXAMPLE 2

### Testing of Example 1 Catalyst in Isobutane/Isooctene Alkylation

The catalyst of Example 1 was used in the alkylation testing of a model feed mixture of isobutane and isooctene having the following composition by weight:

| | |
|---|---|
| iso-C₈= | 2.4% |
| iso-butane | 97.37% |
| n-butane | 0.23% |

The reactor used in these experiments comprised a stainless steel tube having an internal diameter of ⅜ in, a length of 20.5 in and a wall thickness of 0.035 in. A piece of stainless steel tubing 8¾ in. long×⅜ in. external diameter and a piece of ¼ inch tubing of similar length were positioned in the bottom of the reactor (one inside of the other) as a spacer to position and support the catalyst in the isothermal zone of the furnace. A ¼ inch plug of glass wool was placed at the top of the spacer to keep the catalyst in place. A ⅛ inch stainless steel thermo-well was placed in the catalyst bed, long enough to monitor temperature throughout the catalyst bed using a movable thermocouple. The catalyst is loaded with a spacer at the bottom to keep the catalyst bed in the center of the furnace's isothermal zone.

The catalyst was then loaded into the reactor from the top. The catalyst bed typically contained about 4 gm of catalyst sized to 14-25 mesh (700 to 1400 micron) and was 10 cm. in length. A ¼ in. plug of glass wool was placed at the top of the catalyst bed to separate quartz chips from the catalyst. The remaining void space at the top of the reactor was filled with quartz chips. The reactor was installed in the furnace with the catalyst bed in the middle of the furnace at the pre-marked isothermal zone. The reactor was then pressure and leak tested typically at 300 psig (2170 kPa-a).

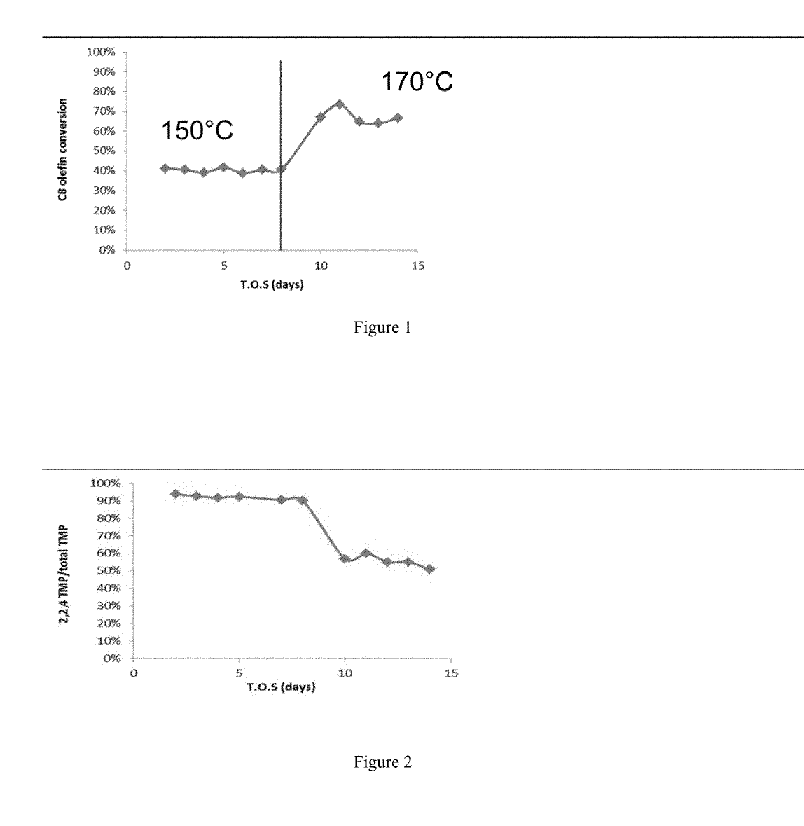

500 cc ISCO syringe pumps were used to introduce the feed to the reactor. Two ISCO pumps were used for pumping the iso-butane (high flow rate 10-250 cc/hr) and one ISCO pump for pumping isooctene (0.1-5 cc/hr). A Grove “Mity Mite” back pressure controller was used to control the reactor pressure typically at 750 psig (5272 kPa-a). On-line GC analyses were taken to verify feed and the product composition. The feeds were then pumped through the reactor with the temperature initially being held at 150° C. and then, after eight days on stream, increased to 170° C. The products exiting the reactor flowed through heated lines routed to GC then to three cold (5-7° C.) collection pots in series. The non-condensable gas products were routed through a gas pump for analyzing the gas effluent. Material balances were taken at 24 hr intervals. Samples were taken for analysis. The material balance and the gas samples were taken at the same time while an on-line GC analysis was conducted for doing material balance. The results of the catalytic testing are summarized in FIGS. 1 and 2.

FIG. 1 shows that the C₈₌ conversion remained substantially constant at around 40% during the first eight days on stream at 150° C. and then increased to around 60-70% when the temperature was increased to 170° C. and then again stayed constant at this higher range for the remaining five days of the test.

FIG. 2 shows that the 2,2,4-dimethylpentane selectivity remained substantially constant at around 90% during the first eight days of the test then decreased to around 60% when the temperature was increased from 150 to 170° C. The 2,2,4-dimethylpentane selectivity showed some further small decrease during the final five days at 170° C. It is to be appreciated that the majority of the trimethyl pentane is formed by alkylation of isobutane in the feed with iso-butylene formed by hydride transfer between the isobutane and isooctene in the feed. These results not only show that heavy (C5+) olefins can be used as alkylating agents over the MWW zeolite, but also these olefins can undergo hydride transfer with isoparaffins to generate the corresponding isoolefins which can further alkylate the isoparaffin feed to produce high octane products, such as 2,2,4-dimethylpentane.

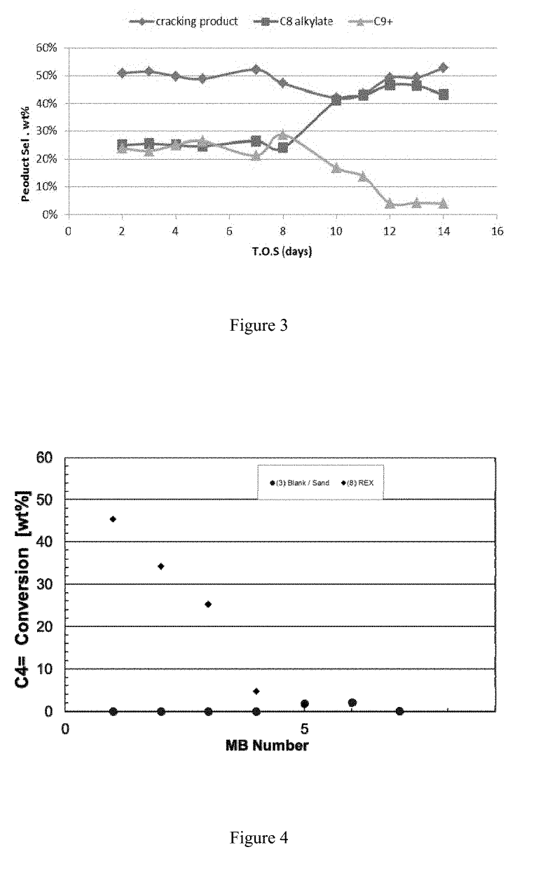

FIG. 3 showing the product selectivity from example 2. The data show that at higher temperature the selectivity to C₉₊ declines dramatically due to the cracking reaction and more C₈ alkylate is formed. Also the cracking product selectivity declined for a while but overall remaining steady. This confirms that recycling heavies improves alkylate yield.

### Example 3

### Testing of REX Catalyst in Isobutane/Isobutene Alkylation

The process of Example 2 was repeated but with the catalyst being REX and the feed being a mixture of isobutane and 2-butene having the following composition by weight:

| | |
|---|---|
| 1-butene | 0.01% |
| Cis-2-butene | 1.25% |
| Trans-2-butene | 1.19% |
| Iso-C₄= | 0.00% |
| Iso-butane | 97.37% |
| n-butane | 0.23% |

The results are shown in FIG. 4 and, when compared with the data in FIG. 1, demonstrate that the REX catalyst was less active and deactivated more rapidly in the alkylation of isobutane with 2-butene than the MCM-49 catalyst when used in the alkylation of isobutane with isooctene.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

1. A process for the catalytic alkylation of an olefin with an isoparaffin, the process comprising:
(a) contacting a feed comprising at least one olefin and at least one isoparaffin with a solid acid catalyst under alkylation conditions effective for reaction between the olefin and the isoparaffin to produce an alkylated product, wherein the solid acid catalyst comprises a crystalline microporous material of the MWW framework type,
(b) separating a C₁₀₊ fraction from the alkylated product; and
(c) recycling at least part of the C₁₀₊ fraction to the contacting (a).

2. The process of claim 1, wherein the feed comprises at least one C₃ to C₁₂ olefin.

3. The process of claim 1, wherein the feed comprises at least one olefin selected from the group consisting of propylene, butenes, pentenes and mixtures thereof.

4. The process of claim 1, wherein the feed comprises at least one C₄ to C₈ isoparaffin.

5. The process of claim 1, wherein at least one of the olefin-containing feed and the isoparaffin-containing feed is pretreated to remove impurities prior to the contacting step.

6. The process of claim 1, wherein the feed comprises isobutane.

7. The process of claim 6, wherein the alkylate product also comprises a C₆ fraction comprising at least 10 wt % of 2,3-dimethylbutane.

8. The process of claim 1, wherein the weight ratio of the C₁₀₊ fraction recycled to the contacting (a) to the feed to the contacting (a) is from 0.1 to 5.

9. The process of claim 1 and further comprising:
(d) recovering a C₉₋ fraction from the alkylated product

10. The process of claim 1, wherein the solid acid catalyst is substantially binder-free.

11. The process of claim 1, wherein the solid acid catalyst comprises an inorganic oxide binder.

12. The process of claim 11, wherein the inorganic oxide binder comprises alumina.

13. The process of claim 11, wherein the inorganic oxide binder is substantially free of amorphous alumina.

14. The process of claim 11, wherein the inorganic oxide binder comprises silica.

15. The process of claim 1, wherein the crystalline microporous material of the MWW framework type is selected from the group consisting of MCM-22, PSH-3, SSZ-25, ERB-1, ITQ-1, ITQ-2, MCM-36, MCM-49, MCM-56, EMM-10, EMM-12, EMM-13, UZM-8, UZM-8HS, UZM-37, MIT-1, and mixtures thereof.

16. The process of claim 1, wherein the crystalline microporous material of the MWW framework type comprises MCM-49.

17. The process of claim 1, wherein the MWW framework type material contains up to 10% by weight of impurities of other framework structures.

18. The process of claim 1, wherein the alkylation conditions include a temperature at least equal to the critical temperature of the principal component of the combined olefin-containing feed and isoparaffin-containing feed and pressure at least equal to the critical pressure of the principal component of the combined olefin-containing feed and isoparaffin-containing feed.
